(19) United States
(12) Patent Application Publication
(10) Pub. No.: US 2002/0123793 A1
(43) Pub. Date: Sep. 5, 2002

(54) Stent including human or animal tissue

(75) Inventors: Max Schaldach, Erlangen (DE); Andreas Becker, Erlangen (DE); Harald Barthel, Erlangen (DE); Max Schaldach JR., Berlin (DE)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co.

(21) Appl. No.: 09/996,061

(22) Filed: Nov. 27, 2001

(30) Foreign Application Priority Data
Nov. 29, 2000 (DE) 100 60 443.9

(51) Int. Cl. A61F 2/06

(52) U.S. Cl. 623/1.15

(57) ABSTRACT

A stent, in particular a coronary stent, comprising a tubular body (2; 2′; 2″) for expansion from a first condition into a second condition in which it holds a vessel (18) of the human or animal body in an expanded state, wherein the tubular body (2; 2′; 2″) includes at least a first wall portion (4; 4′; 4″) comprising a human or animal tissue of adequate elasticity.

The present invention concerns a stent, in particular a coronary stent, comprising a tubular body for expansion from a first condition into a second condition in which it holds a vessel in the human or animal body expanded. It further concerns a catheter for implanting a stent and a process for producing a stent.

### BACKGROUND OF THE ART

Such stents which are frequently also referred to as intraluminal expansion elements generally served to hold a blood vessel which is constricted for example as a result of arteriosclerosis in an expanded state and thus to restore the normal function thereof. They are however also used in the area of other vessels in the human or animal body in order to treat stenoses, that is to say constrictions. In that respect, they are generally introduced into the vessel in a compressed first condition by means of a catheter, moved in the vessel to the implantation location, and then expanded in the region thereof into the second condition in which they hold the vessel in an expanded state.

Stents of that kind however can also be used for bridging over weak points in the vessel, as occur for example in the region of aneurysms. In that case the stents serve at least for anchoring the prosthesis which bridges over the weak point, at both sides of the weak points. They can however themselves also form the prosthesis, with a suitable closed wall configuration.

A distinction is made between what are referred to as balloon-expansible stents which are arranged on a balloon and which are expanded by same into the second condition, and what are referred to as self-expanding stents. The latter, in being moved to the implantation location, are arranged in a sheath which holds them compressed to a reduced diameter. At the implantation location, the sheath is removed and the stent expands of its own accord into its expanded second condition, by virtue of the elastic deformation energy stored in the stent.

In that respect conventional stents generally comprise metal or metal alloys. It is equally known for stents of that kind to be produced from plastic materials or composite materials. In order to prevent what are referred to as re-stenoses or other reactions in respect of the vessel, it is known for the stents to be provided with coatings of body-specific or human substances or medicament-bearing coatings.

The known stents however suffer on the one hand from the disadvantage that under some circumstances the materials used can give rise to more less severe immune reactions on the part of the patient. They can manifest themselves in inflammation and other possibly threatening damage to the vessel.

A further disadvantage lies in the flexibility of the materials used hitherto. Particularly in the case of stents of metal, of a grid or mesh form, when they are being moved to the implantation location through vessel configurations which in part are heavily curved, there is the danger of individual grid or mesh elements failing or at least being so adversely affected that they fail upon subsequent expansion. Under some circumstances that gives rise to sharp edges which can represent a risk factor for the vessel.

An additional disadvantage of the known stents lies in the relatively expensive manufacture of such stents. Thus the mesh structures of the metal stents are frequently produced from suitable metal tube portions by means of expensive laser cutting processes and so forth.

Therefore the object of the present invention is to provide a stent of the kind set forth in the opening part of this specification, which does not suffer from the above-indicated disadvantages or suffers therefrom at least to a lesser degree, and which in particular can be particularly easily implanted with good compatibility.

### SUMMARY OF THE INVENTION

Based on a stent as set forth in the classifying portion of claim 1 that object is attained by the features recited in the characterizing portion of claim 1.

The present invention is based on the technical teaching that a stent which is particularly body-compatible and simple to implant is obtained if the tubular body includes at least a first wall portion of human or animal tissue of adequate elasticity.

The first wall portion preferably extends over the entire length of the stent. In that case it can form the entire tubular body. The first wall portion however can also form only a part of the tubular body. Thus, it can be envisaged for example that a plurality of wall portions of the stent are in mutually interleaved relationship. It is equally possible for different wall portions to alternate with each other over the length of the stent.

In this case the wall portions may comprise different materials. In that respect the first wall portion may be combined with wall portions comprising conventional stent materials. In that case the first wall portion then preferably forms the wall portion which directly adjoins the vessel in order to ensure that the vessel wall is contacted as much as possible with a material which is highly body-compatible. In those variants, the first wall portion is preferably combined with wall portions which also comprise a suitable human or animal tissue.

The stents according to the invention have on the one hand the advantage that, while affording good to very good compatibility with the body, they generally enjoy adequate elasticity to be able to be moved to the implantation location without damage even through very severely curved vessel configurations.

A further advantage is that the wall portions of human or animal tissue can be easily cultivated in known manner, for example in the form of cell cultures, in a suitable nutrient solution. The production thereof is therefore a particularly simple matter. In that respect, it is possible in particular to produce any stent geometries for example by virtue of a suitable configuration for the carrier of the culture. The wall of the stent itself may also be of any desired configuration. It may be of any apertured structure or mesh structure. It will be appreciated however that the material used also makes it possible to embody a stent with a completely closed wall, which has an advantageous effect in terms of preventing re-stenoses.

A further advantage lies in the body compatibility which can be achieved. Thus it is possible for example for the stent to be produced from body-specific material of the patient to be treated, so that immune reactions are excluded from the outset. Equally however, with comparably good compatibility, it is also possible for the stent to be produced from human or animal tissue which has been suitably treated and possibly genetically modified.

The stiffness of the stent, which is required to hold the vessel in the expanded state, can be achieved in various ways. Thus preferred variants of the stent according to the invention are distinguished in that the first wall portion of itself is of a stiffness which is sufficient to hold the vessel expanded, in the second condition. In this case therefore the human or animal tissue used itself must already be of sufficient stiffness. For that purpose it is possible to use any suitable tissue which has possibly been treated or modified in the above-mentioned fashion.

Particularly good results are achieved with preferred variants of the stent according to the invention in which the first wall portion comprises cartilage tissue. In that respect, depending on the respective situation of use, it is possible to use the different types of cartilage. For example what is referred to as hyaline cartilage is suitable for highly loaded stents. If a particularly high degree of elasticity is required it is possible for example to use what is referred to as elastic cartilage. Fibrous or connective tissue cartilage can also advantageously be used.

As already mentioned above the human or animal tissue employed can be genetically modified. The modification is preferably to the effect that the body compatibility of the tissue is increased. Additionally or alternatively the modification can also provide that the stiffness of the tissue is increased. Likewise it is possible to so select the modification as to achieve an increased service life for the stent.

In other variants of the stent according to the invention the stiffness of the material of the first wall portion in itself is not sufficient to hold the vessel expanded, in the second condition of the stent. In that case additional measures are then required to achieve the adequate degree of stiffness.

Thus for example in the case of preferred variants of the stent according to the invention it is provided that the first wall portion comprises a hardenable tissue in order to produce the required stiffness for the wall portion, by virtue of using a suitable hardener. That tissue is preferably permeable in relation to the biocompatible hardener in order in that way to achieve hardening which is as uniform as possible of the entire tissue. The term hardening is used in accordance with the present invention to denote stiffening of the tissue. After hardening in the sense of the present invention the tissue preferably still has at least such a high degree of elasticity that the stent can still perform the natural movements of the vessel.

Hardening of the tissue can be effected in different ways. Thus for example it is possible to provide a hardening agent which hardens at body temperature. The stent then has to be held at a lower temperature between application of the hardening agent to the first wall portion and expansion of the stent, in order thereby to prevent premature hardening. Likewise it is possible to provide hardening agents which harden under the effect of irradiation for example with laser light and so forth. It can further be provided that the hardening agent is applied to the stent only upon or shortly before positioning thereof at the appropriate location in the vessel.

It will be noted however that one or more components of the hardening agent may already be applied to the stent long before that moment in time and then the component or components which initiate the hardening reaction is or are applied shortly before or upon positioning of the stent.

Thus, in preferred variants, the stent according to the invention is so designed that the first wall portion is at least in a portion-wise manner provided with at least a first layer which includes at least a first component of a hardening agent. Likewise the first wall portion can include at least in a portion-wise manner at least a first component of a hardening agent. Thus for example it can be impregnated with a liquid or the like which contains the first component of the hardening agent. The first component however may also be introduced into the wall portion in another fashion. Thus the first component may already be introduced into the wall portion for example during production thereof, for example by virtue of being distributed in the nutrient solution.

In this arrangement, at least the first component of the hardening agent may be enclosed in microcapsules which burst open under the effect of pressure. That variant is suitable in particular in combination with variants in which expansion of the stent into the second condition is effected by means of a balloon catheter. In that situation the pressure applied to the stent by the balloon upon expansion causes the microcapsules to burst open so that the first component of the hardening agent, which is contained therein, is liberated, and the hardening reaction is then initiated with one or more further components which are present outside the microcapsules. It will be appreciated that, in the case of other, in particular self-expanding variants of the stent, the pressure which acts between the vessel and the stent upon expansion into the second condition can be sufficient to cause the microcapsules to burst. It will be further appreciated that, in preferred variants, the further component or further components of the hardening agent are also enclosed in such microcapsules.

In preferred variants of the stent according to the invention with wall portions which are in interleaved or interlocking relationship, that is to say with at least a second wall portion which is arranged in the first wall portion in the second condition of the stent, it is provided that the first layer is arranged on the surface which is towards the second wall portion and the second wall portion is provided at least in a portion-wise manner, on its surface which is towards the first wall portion, with at least a second layer which includes at least a second component of the hardening agent. Depending on the respective reaction time of the components of the hardening agent, that is to say depending on how long the stent geometry still remains capable of change after the components have been brought into contact with each other, the first and second wall portions are possibly introduced into each other only shortly before expansion of the stent, that is to say possibly also at the implantation location. In this case also however the above-mentioned capsules can once again be used to advantage.

In other variants of the stent according to the invention the stiffness required for holding the vessel open is additionally or alternatively to the above-mentioned options achieved by gluing to an element adjoining the first wall portion in the second condition of the stent. In the case of variants which have mutually interleaved wall portions the adjoining element may be for example a further wall portion of the stent. The adjoining element may also be formed by the vessel itself, into which the stent is fitted.

For that purpose the stent according to the invention is preferably so designed that the first wall portion, to produce an adhesive join to an element which adjoins it in the second condition is provided at least in a portion-wise manner with at least a third layer which includes at least a first component of an adhesive. The required stiffness of the composite arrangement can be achieved in that case inter alia by virtue of the resulting adhesive layer comprising the adhesive being of adequate shearing stiffness. Likewise the composite arrangement comprising a plurality of mutually interleaved wall portions may achieve adequate stiffness by virtue of the adhesive join.

Hardening or setting of the adhesive can again be effected in different ways. It is thus possible for example to provide an adhesive which hardens at body temperature. In that case the stent must be kept at a lower temperature between application of the adhesive to the first wall portion and expansion of the stent, in order to prevent the adhesive from prematurely hardening or setting. It is likewise possible to provide adhesives which harden or set under irradiation, for example with laser light and so forth. It can further be provided that the adhesive is applied to the stent only upon or shortly before positioning thereof at the appropriate location in the vessel.

It will be noted that one or more components of the adhesive may also already be applied to the stent long before that moment in time and that then the component or components which initiate the hardening reaction is or are applied shortly before or upon positioning of the stent.

Thus, in preferred variants, the stent according to the invention is of such a nature that the first wall portion is provided at least in a portion-wise manner with at least a third layer which includes at least a first component of an adhesive. Likewise the first wall portion may include at least in a portion-wise manner at least a first component of an adhesive. Thus for example it can be impregnated with a liquid or the like which contains the first component of the adhesive. The first component however can also be introduced into the wall portion in another fashion. Thus for example the first component can already be introduced into the wall portion for example during production of the wall portion, for example by distribution in the nutrient solution.

In that respect at least the first components of the adhesive can be enclosed in microcapsules which burst open under the effect of pressure. This variant is suitable in particular in combination with variants in which expansion of the stent into the second condition is effected by means of a balloon catheter. In that situation the pressure applied to the stent by the balloon upon expansion causes the microcapsules to burst open so that the first component of the adhesive, which is contained therein, is released, and then the hardening reaction is initiated with one or more further components which are present outside the microcapsules. It will be appreciated that in the case of other and in particular self-expanding variants of the stent the pressure which acts between the vessel and the stent upon expansion into the second condition can suffice to cause the microcapsules to burst open. It will further be appreciated that, in preferred variants, the further component or components of the adhesive are enclosed in such microcapsules.

In preferred variants of the stent according to the invention with mutually interleaved wall portions, that is to say with at least a second wall portion arranged in the first wall portion in the second condition of the stent, it is provided that the third layer is arranged on the surface which is towards the second wall portion and the second wall portion is provided, on its surface towards the first wall portion, at least in a portion-wise manner, with at least a fourth layer which includes at least a second component of the adhesive. Depending on the respective reaction time of the components of the adhesive, that is to say depending on how long the stent geometry still remains variable, after the components have been brought into contact with each other, the first and second wall portions are introduced into each other possibly only shortly before expansion, that is to say possibly also only at the implantation location.

In this case also however once again the above-mentioned microcapsules can be used to advantage.

In advantageous embodiments of the stent according to the invention the first wall portion is formed by a flat element which at least in the first condition is rolled up in the manner of a sheet. That is then unrolled upon expansion to a larger diameter and in that position or configuration fixed either by the above-described hardening and/or adhesive join to an adjoining element, for example the vessel itself. Likewise it is possible for mutually overlapping portions of the flat element to be joined together, for example by adhesive as described above.

In this respect the length of the flat element in the peripheral direction of the stent preferably corresponds substantially at least to the periphery of the first wall portion in the second condition. This provides that the ends of the flat element, which are disposed in the peripheral direction of the stent, butt against each other or overlap each other, to assist with the locking action in that second expanded condition, in which case they can then be glued together in the overlap region or joined in some other fashion.

The present invention further concerns a catheter for implantation of a stent, in particular a stent according to the invention, comprising a distal end region, in the region of which are arranged a holding device for holding the stent and a sheathing device which is movable relative to the holding device in the longitudinal direction of the catheter to receive the stent when being moved to the implantation location. When it has arrived at the implantation location, in the case of this catheter, the sheathing device is then retracted in the proximal direction with respect to the stent which is held by the holding device. In that situation the stent can then expand or be expanded into its second condition. In accordance with the invention, provided on the sheathing device is at least one application device for applying to the surface of the stent a medium which is capable of flow.

With that catheter according to the invention it is easily possible, as described above, to apply a hardening agent and/or an adhesive or one or more components of one of those agents to the stent only shortly before expansion thereof at the implantation location. It will be appreciated however that any other agents, for example medicaments or the like, can also be applied to the stent in that way. At any event this affords the advantage that agents which are sensitive or react with other reaction partners and which are to be applied to the stent can be applied thereto only shortly before expansion of the stent and thus prior to expansion are exposed to undesirable environmental influences only for a very short time.

In that respect the medium which is capable of flow can be provided in a suitable storage means at the distal end of the catheter. It is however also possible to provide a feed conduit of suitable length, by way of which the medium is conveyed towards the distal end of the catheter. In variants which are preferred by virtue of their simplicity of design, the application device has at least one application opening in the sheathing device, which opening is connected to a feed passage for the medium which is capable of flow, in particular a component of a hardening agent or adhesive. In that case the application opening is so designed that the medium which is capable of flow is applied to the stent directly in the desired manner of distribution.

The present invention further concerns a catheter for implantation of a stent comprising a distal end region, in the region of which are arranged a holding device for holding the stent and a sheathing device which is movable relative to the holding device in the longitudinal direction of the catheter to receive the stent when being moved to the implantation location. In accordance with the invention in that respect it is provided that the sheathing device is adapted to receive a stent which, on its surface towards the sheathing device, is provided with a layer of an adhesive, wherein, on its surface towards the coated surface of the stent, the sheathing device is provided with an anti-adhesion coating. In that way the stent can be moved in a simple fashion together with the adhesive already applied thereto, to the implantation location, and then the sheathing device can be removed without the adhesive layer being adversely affected by.

In the case of the catheters according to the invention the holding device can be of any desired configuration. It may then be for example in the form of a simple abutment which holds the stent only in a direction towards the proximal end and which ensures that the sheathing device can be drawn off the stent in the proximal direction. It will be appreciated that likewise the holding device can also fix the stent in other directions. In preferred embodiments of the catheter according to the invention the holding device includes a balloon for expanding the stent into a second condition in which it holds a vessel in the human or animal body in an expanded state. That advantageously affords functional integration.

The present invention further concerns a process for producing a stent, in particular a coronary stent, comprising a tubular body for expansion from a first condition into a second condition in which it holds a vessel in the human or animal body in an expanded state. In accordance with the invention at least a first wall portion of the tubular body is made from human or animal tissue cells.

In preferred variants of the process according to the invention the tissue cells, for producing the first wall portion, are cultivated in a shaping mold corresponding to the configuration of the first wall portion or on a corresponding carrier. That makes it possible to produce any desired stent geometries. In particular, with the availability of corresponding geometrical data for the implantation location in the vessel, it is possible for the stent to be produced so-to-speak tailor-made for the use thereof. It is only necessary to produce a suitable shaping mold or a suitable carrier in or on which the cell culture can then grow.

### BRIEF DESCRIPTION OF THE DRAWINGS

Further preferred configurations of the present invention will be apparent from the appendant claims and the description hereinafter of preferred variants of the invention, with reference to the accompanying drawings in which:

FIG. 1 is a diagrammatic view in cross-section through an arrangement comprising a preferred variant of the stent according to the invention in a sheathing catheter,

FIG. 2 is a diagrammatic view in longitudinal section of a preferred embodiment of the stent according to the invention,

FIG. 3 is a diagrammatic view of a detail from the embodiment of FIG. 2,

FIG. 4A is a diagrammatic view in cross-section through a further preferred embodiment of a stent according to the invention in its first condition,

FIG. 4B is a diagrammatic view in cross-section through the stent of

FIG. 4A in its second condition, and

FIG. 5 is a diagrammatic view in longitudinal section through a preferred variant of the catheter according to the invention.

### DETAILED DESCRIPTION OF THE INVENTION

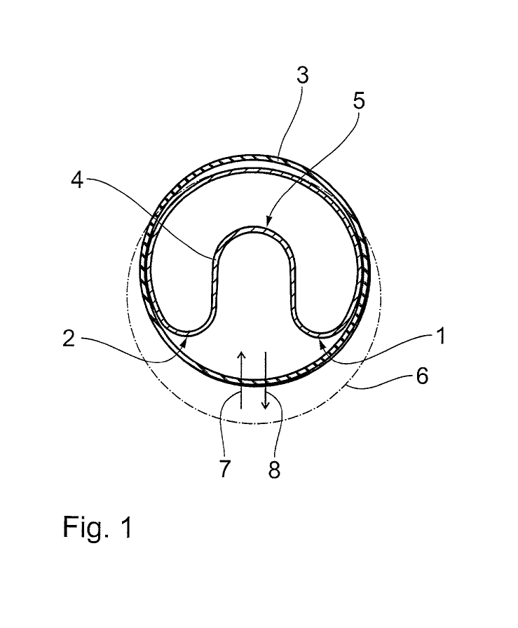

FIG. 1 shows a diagrammatic view in cross-section through a stent 1 according to the invention comprising a tubular body 2 in its first condition in which it can be introduced into a vessel in a human or animal body. The stent 1 in this case is arranged in a sheathing catheter 3.

The stent 1 comprises over its entire length and its entire periphery a first wall portion 4. That wall portion 4 comprises human cartilage tissue. In this case, hyaline cartilage which is distinguished by a particularly high level of stiffness was used for the wall portion 4. It will be appreciated however that other variants may also use other, possibly softer kinds of cartilage.

The thickness of the first wall portion 4 in this case is so selected that the cartilage tissue used affords sufficient stiffness to hold a blood vessel into which the stent 1 is introduced and in which the stent 1 is expanded into its second condition in an expanded state in known manner.

The stent 1 which is elastic by virtue of the cartilage tissue used is folded radially inwardly in the direction of the arrow 7 in its first condition as shown in FIG. 1, in a peripheral region 5, in relation to its unstressed geometry as indicated by the contour 6. In that situation, it is held at a reduced diameter, by the sheathing catheter 3, in opposition to the resilient return forces acting in the stent.

When the sheathing catheter 3 is removed the stent 1 unfolds possibly—with assistance by a suitable instrument—in the direction of the arrow 8 again, by virtue of the resilient return forces. In doing so it then bears against the wall of the vessel (not shown) into which it is introduced, in which case the resilient return forces acting therein are sufficient to hold that vessel in the expanded state.

The wall portion 4 of the stent 1 was produced by cultivation of suitable cartilage cells taken from the patient in question. In that case the cartilage cells would be cultivated in a nutrient solution in a shaping mold whose cultivation space substantially corresponds to the later configuration of the wall portion 4. The stent 1 only had to be processed at its two ends, in which case it was only cut to the desired length.

It will be appreciated that other variants may also use human tissue which was not taken from the patient in question but elsewhere. That tissue is then preferably modified by genetic modification in such a way that it does not cause any immune reactions. Likewise however it is also possible to use animal tissue which is then also preferably genetically modified in a suitable manner.

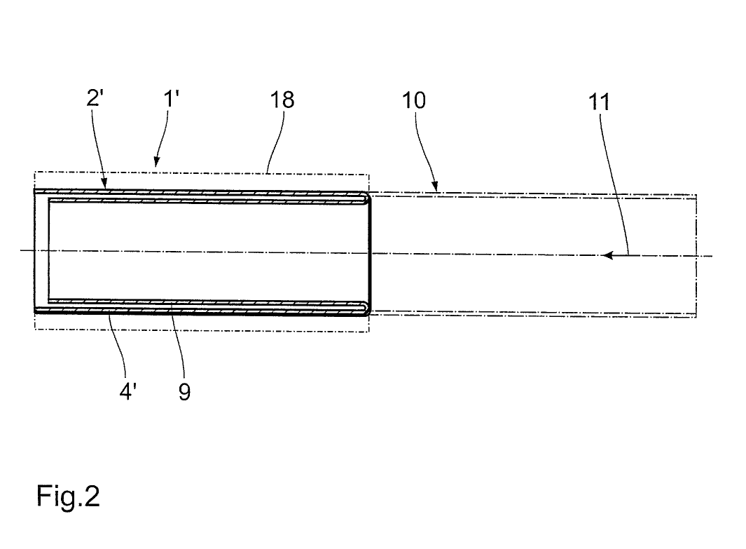

FIG. 2 shows a diagrammatic view in longitudinal section through a further embodiment of the stent 1′ according to the invention with a tubular body 2′ comprising a first wall portion 4′ and a second wall portion 9 which are interleaved with each other. In this arrangement the second wall portion 9 is disposed in the interior of the first wall portion 4′.

In the starting condition of the stent 1′ the first wall portion 4′ and the second wall portion 9 adjoin each other in the longitudinal direction of the stent, as is indicated by the contour 10. So that the two wall portions 4′ and 9 are interleaved with each other, the wall portion 9 is pulled into the wall portion 4′ in the direction indicated by the arrow 11. That is possible by virtue of the elasticity of the human tissue which here too is used for the wall portions. In this case the tissue used is softer than the cartilage tissue described in relation to FIG. 1. It will be noted however that in this case also it is possible to use cartilage tissue.

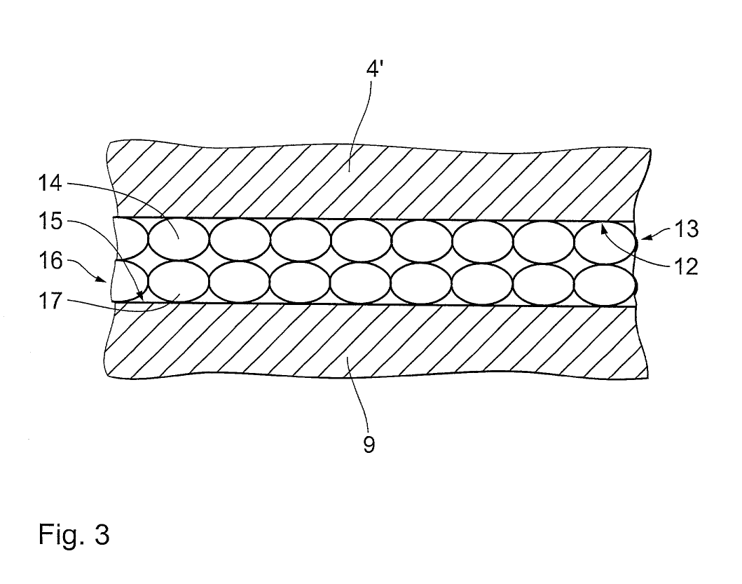

As can be seen from FIG. 3, in this case a first layer 13 of microcapsules 14 is provided on the surface 12 of the first wall portion 4′, which in the interleaved condition faces towards the second wall portion 9. In addition, a second layer 16 of microcapsules 17 is arranged on the surface 15 of the second wall portion 9, which in the interleaved condition faces towards the first wall portion 4′. In this case the microcapsules 14 and 17 contain the two components, which are capable of flow, of a hardening agent.

When the stent 1′ is expanded by means of a balloon catheter (not shown) into its second condition as indicated in FIG. 2 by the dash-dotted contour 18, the microcapsules 14 and 17 burst open under the effect of pressure. In that way the two components of the hardening agent are liberated and are mixed together so that the hardening reaction begins. At the same time, assisted by the action of pressure, the hardening agent penetrates into the wall portions 4′ and 9 which are capable of absorbing the hardening agent so that ultimately at least a considerable proportion of the wall portions is permeated with the hardening agent.

The pressure applied by the balloon (not shown) is maintained until the hardening agent has sufficiently hardened to ensure that the stent remains in its second condition. In this respect, the combination of hardening agent and tissue for the wall portions 4′ and 9 is so selected that on the one hand the stent 1′ enjoys sufficient stiffness to hold the vessel into which it is introduced in the expanded state. On the other hand, in the illustrated example, in that second state, the stent still has sufficient flexibility to permit natural movements of the vessel.

It will be appreciated that, in other variants, the microcapsules may for example also contain the components of an adhesive, which components are then liberated upon expansion of the stent. In that case, an adhesive join is then formed between the two mutually adjoining wall portions, and that can be sufficient to ensure sufficient stiffness for the stent. In that case, the adhesive itself can form an intermediate layer which has sufficient shearing strength and which is firmly joined to the adjoining wall portions and which possibly crucially contributes to the stiffness of the stent. It will be appreciated moreover that, in other advantageous embodiments of the invention, a combination of hardening and adhesive join is also possible.

It will further be appreciated that the illustrated hardening effect can be used not only in connection with wall portions in mutually interleaved relationship. Thus, it can also be used in connection with a single first wall portion. The microcapsules with the first component and the microcapsules with the second component can then be disposed for example on the surface of the first wall portion, which is towards the balloon.

It will further be appreciated that the components of the hardening agent or the adhesive do not necessarily have to be enclosed in such microcapsules. They may also be arranged on or applied to the wall portion, in exposed layers. It is then only necessary to consider the times which, after the components are brought into contact, are still available for further changing the stent geometry.

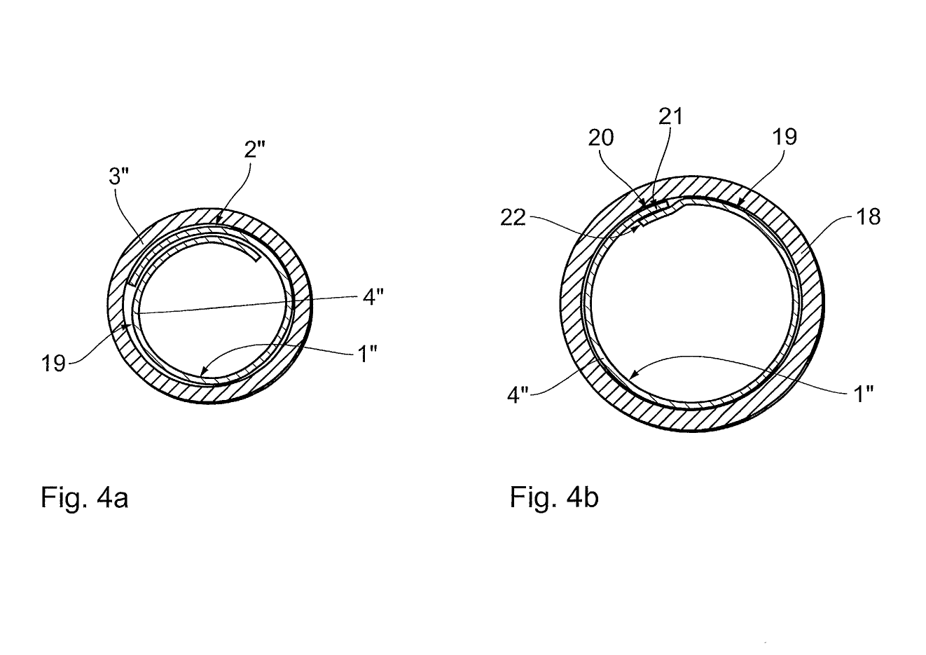

FIGS. 4A and 4B show a further variant of the stent 1″ according to the invention. This is shown in FIG. 4A in its compressed first condition in which it is arranged in a sheathing catheter 3″.

The stent 1″ comprises a tubular body 2″ comprising a first wall portion which in turn is formed by a flat element 4″ which is rolled up in the manner of a sheet. In this case the element 4″ comprises animal tissue which is genetically modified to enhance body compatibility. It will be appreciated however that in other variants it is also possible to use human tissue, in particular cartilage tissue.

FIG. 4B shows the stent 1″ in its expanded second condition in which it holds a blood vessel 18 in an expanded state. It is expanded into that second condition after removal of the sheathing catheter 3″ by a balloon catheter (not shown).

On its outside 19 the element 4″ is provided with microcapsules (not shown) which contain in part the first component of an adhesive and in part the second component of the adhesive. Due to the pressure action of the balloon upon expansion or the counteracting pressure exerted by the blood vessel 18 the microcapsules burst open and, after a certain hardening time, the adhesive forms a strong join between the element 4″ and the blood vessel 18.

In this case the length of the flat element 4″ in the peripheral direction of the stent 1″ is so selected that it exceeds the periphery of the stent 1″ in the illustrated second condition so that there is an overlap 20, in the region of which the two ends 21 and 22, which face in the peripheral direction, of the flat element 4″ are glued together. Both by virtue of the gluing of those two ends 21 and 22 and also by virtue of the gluing of the flat element 4″ to the vessel 18, the result achieved is a sufficiently stiff composite arrangement which ensures that the vessel 18 is held in an expanded condition and is possibly also sealed off.

It will be appreciated that, in other variants, the elasticity and stiffness of the tissue used for the flat element can be so selected that no balloon is required to expand the stent, but the stent can expand solely by virtue of the resilient return forces acting therein, after removal of the sheathing catheter.

It will further be appreciated that, in other variants, in this case also a hardening agent or a combination of hardening agent and adhesive may again be used in the above-described manner.

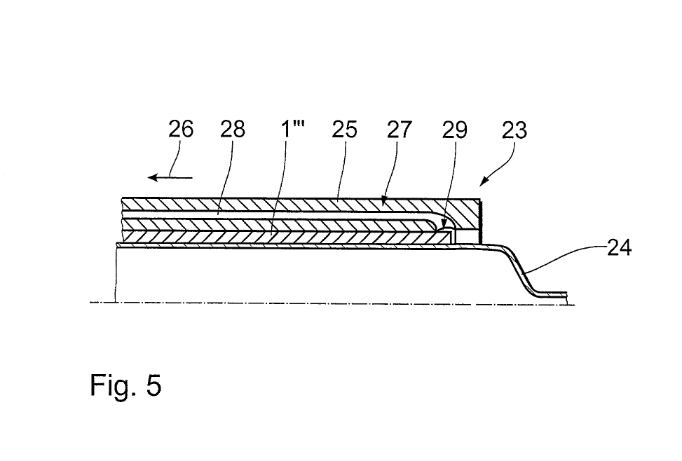

FIG. 5 shows a view in longitudinal section through the distal end of an embodiment of the catheter 23 according to the invention, with a holding device for the stent 1′″, formed by a balloon 24, and a sheathing device formed by a sheathing tube 25. The sheathing tube 25 is displaceable in the longitudinal direction of the stent 1′″ with respect to the balloon 24.

When the sheathing tube 25 is retracted in the proximal direction in the direction indicated by the arrow 26, the stent 1′″ which is shown in FIG. 5 in its compressed first condition can be expanded into its second expanded condition in known manner by means of the balloon 24,

When the sheathing tube 25 is withdrawn in the proximal direction, a component which is capable of flow of a hardening agent is applied by way of an applicator device 27 which has feed passages 28 and an application opening 29 extending in an annular configuration over the inner periphery of the sheathing tube. The flowable component of the hardening agent reacts with a further component thereof with which the stent 1′″ is impregnated.

Withdrawal of the sheathing tube 25 and application of the component of the hardening agent are effected immediately prior to expansion of the stent 1′″ by the balloon 24. In that case the hardening agent is so selected that it hardens within a few minutes in order to minimize the implantation time.

It will be appreciated that, in other variants of the catheter according to the invention, it can also be provided that an adhesive layer is already applied on the outside of the stent in the first condition, when the sheathing tube has not yet been withdrawn. The sheathing tube is then provided with an anti-adhesion coating on its side which is towards the stents.

1. A stent, in particular a coronary stent, for a vessel of a human or animal body, comprising:
a tubular body for expansion from a first condition into a second condition in which it holds the vessel in an expanded state, wherein includes at least a first wall portion comprising a human or animal tissue of adequate elasticity.

2. The stent of claim 1, wherein the first wall portion has a stiffness which is adequate to hold the vessel in the expanded state in the second condition.

3. The stent of claim 1, wherein the first wall portion comprises cartilage tissue.

4. The stent of claim 1, wherein the first wall portion comprises a tissue which is genetically modified to increase compatibility and/or stiffness.

5. The stent of claim 1, wherein the first wall portion comprises a hardenable tissue.

6. The stent of claim 5, wherein the first wall portion is provided in at least a portion wise manner with at least a first layer which includes at least a first component of a hardening agent or at least in a portion-wise manner contains at least a first component of a hardening agent.

7. The stent of claim 5, further comprising: a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

8. The stent of claim 5, wherein at least the first component of the hardening agent is enclosed in microcapsules which burst open under the effect of pressure.

9. The stent of claim 1, wherein the first wall portion is provided at least in a portion-wise manner with at least a third layer which includes at least a first component of an adhesive or contains at least in a portion-wise manner at least a first component of an adhesive, to produce an adhesive join to an element adjoining the first wall portion in the second condition.

10. The stent of claim 9, wherein a second wall portion is provided which is arranged in the first wall portion at least in the second condition of the stent, wherein the third layer is arranged on the surface towards the second wall portion and the second wall portion is provided on its surface towards the first wall portion, at least in a portion-wise manner, with at least a fourth layer which includes at least a second component of the adhesive.

11. The stent of claim 9, wherein at least the first component of the adhesive is enclosed in microcapsules which burst open under the effect of pressure.

12. The stent of claim 1, wherein the first wall portion is formed by a flat element (4″) which is rolled up in the manner of sheet at least in the first condition.

13. The stent of claim 12, wherein the flat element has a length in a peripheral direction of the stent that corresponds substantially at least to a periphery of the first wall portion in the second condition.

14. A catheter for implanting a stent as set forth in one of claim 1, comprising:
a distal end region; a holding device for holding the stent, arranged near the distal end region; and
a sheathing device, also near the distal end region, which is movable relative to the holding device in a longitudinal direction of the catheter for receiving the stent when moving it to an implantation location, characterized in that at least one application device is provided at the sheathing device for applying a medium which is capable of flow to a surface of the stent.

15. The catheter of claim 14, wherein the application device further comprises at least one application opening in the sheathing device (25), which opening is connected to a feed passage for the medium which is capable of flow.

16. A catheter for implanting a stent as set forth in one of claim 1 comprising:
a distal end region;
a holding device for holding the stent, arranged near the distal end region; and a sheathing device, also near the distal end region, which is movable relative to the holding device in a longitudinal direction of the catheter for receiving the stent when moving it to an implantation location, characterized in that the sheathing device received the stent which has a layer of adhesive coated on its surface towards the sheathing device, which has an anti-adhesion coating on its surface toward the coated stent surface

17. The catheter of claim 14, wherein the holding device further comprises includes a balloon (24) for expansion of the stent into a second condition in which it holds a vessel in a human or animal body in an expanded state.

18. The catheter of claim 17, further comprising:
a stent as set forth in claim 1.

19. A process for producing a stent, in particular a coronary stent, comprising a tubular body for expansion from a first condition into a second condition in which it holds a vessel in the human or animal body in the expanded state, characterized in that at least a first wall portion of the tubular body is produced from human or animal tissue cells.

20. The process of claim 19, wherein the tissue cells are cultivated in a shaping mold corresponding to the configuration of the first wall portion or on a corresponding carrier to produce the first wall portion.

21. (new) The stent of claim 2, wherein the first wall portion comprises cartilage tissue.

22. (new) The stent of claim 2, wherein the first wall portion comprises a tissue which is genetically modified to increase compatibility and/or stiffness.

23. (new) The stent of claim 3, wherein the first wall portion comprises a tissue which is genetically modified to increase compatibility and/or stiffness.

24. (new) The stent of claim 21, wherein the first wall portion comprises a tissue which is genetically modified to increase compatibility and/or stiffness.

25. (new) The stent of claim 2, wherein the first wall portion comprises a hardenable tissue.

26. (new) The stent of claim 23, wherein the first wall portion comprises a hardenable tissue.

27. (new) The stent of claim 4, wherein the first wall portion comprises a hardenable tissue.

28. (new) The stent of claim 24, wherein the first wall portion comprises a hardenable tissue.

29. (new) The stent of claim 22, wherein the first wall portion comprises a hardenable tissue.

30. (new) The stent of claim 25, wherein the first wall portion is provided in at least a portion wise manner with at least a first layer which includes at least a first component of a hardening agent or at least in a portion-wise manner contains at least a first component of a hardening agent.

31. (new) The stent of claim 26, wherein the first wall portion is provided in at least a portion wise manner with at least a first layer which includes at least a first component of a hardening agent or at least in a portion-wise manner contains at least a first component of a hardening agent.

32. (new) The stent of claim 27, wherein the first wall portion is provided in at least a portion wise manner with at least a first layer which includes at least a first component of a hardening agent or at least in a portion-wise manner contains at least a first component of a hardening agent.

33. (new) The stent of claim 28, wherein the first wall portion is provided in at least a portion wise manner with at least a first layer which includes at least a first component of a hardening agent or at least in a portion-wise manner contains at least a first component of a hardening agent.

34. (new) The stent of claim 29, wherein the first wall portion is provided in at least a portion wise manner with at least a first layer which includes at least a first component of a hardening agent or at least in a portion-wise manner contains at least a first component of a hardening agent.

35. (new) The stent of claim 6, further comprising:
a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

36. (new) The stent of claim 30, further comprising:
a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

37. (new) The stent of claim 31, further comprising:
a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

38. (new) The stent of claim 32, further comprising:
a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

39. (new) The stent of claim 33, further comprising:
a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

40. (new) The stent of claim 34, further comprising:
a second wall portion arranged in the first wall portion at least in the second condition of the stent, wherein the first layer is arranged on the surface which is towards the second wall portion and the second wall portion, on its surface towards the first wall portion, is provided at least in a portion-wise manner with at least a second layer which includes at least a second component of the hardening agent.

41. (new) The stent of claim 6, wherein at least the first component of the hardening agent is enclosed in microcapsules which burst open under the effect of pressure.

42. (new) The stent of claim 7, wherein at least the first component of the hardening agent is enclosed in microcapsules which burst open under the effect of pressure.

43. (new) The stent of claim 39, wherein at least the first component of the hardening agent is enclosed in microcapsules which burst open under the effect of pressure.

44. (new) The stent of claim 43, wherein the first wall portion is provided at least in a portion-wise manner with at least a third layer which includes at least a first component of an adhesive or contains at least in a portion-wise manner at least a first component of an adhesive, to produce an adhesive join to an element adjoining the first wall portion in the second condition.

45. (new) The stent of claim 9, wherein a second wall portion is provided which is arranged in the first wall portion at least in the second condition of the stent, wherein the third layer is arranged on the surface towards the second wall portion and the second wall portion is provided on its surface towards the first wall portion, at least in a portion-wise manner, with at least a fourth layer which includes at least a second component of the adhesive.

46. (new) The stent of claim 10, wherein at least the first component of the adhesive is enclosed in microcapsules which burst open under the effect of pressure.

47. (new) The stent of claim 44, wherein at least the first component of the adhesive is enclosed in microcapsules which burst open under the effect of pressure.

48. (new) The stent of claim 45, wherein at least the first component of the adhesive is enclosed in microcapsules which burst open under the effect of pressure.

49. (new) The stent of claim 48, wherein the first wall portion is formed by a flat element which is rolled up in the manner of sheet at least in the first condition.

50. (new) The stent of claim 49, wherein the flat element has a length in a peripheral direction of the stent that corresponds substantially at least to a periphery of the first wall portion in the second condition.

51. (new) The catheter of claim 16, wherein the holding device further comprises a balloon for expansion of the stent into a second condition in which it holds a vessel in a human or animal body in an expanded state.

52. (new) The catheter of claim 15, wherein the holding device further comprises a balloon for expansion of the stent into a second condition in which it holds a vessel in a human or animal body in an expanded state.
